(19) United States
(12) Patent Application Publication
(10) Pub. No.: US 2005/0159903 A1
(43) Pub. Date: Jul. 21, 2005

(54) Radiographic apparatus, and control method and program thereof

(76) Inventor: Takashi Ogura, Tokyo (JP)

Correspondence Address: MORGAN & FINNEGAN, L.L.P., 3 WORLD FINANCIAL CENTER, NEW YORK, NY, 10281-2101, US

(21) Appl. No.: 11/039,470

(22) Filed: Jan. 19, 2005

(30) Foreign Application Priority Data
Jan. 21, 2004 (JP) 2004-013106

(51) Int. Cl. G01B 5/28; G06F 19/00; G01B 5/30; G01N 23/04

(52) U.S. Cl. 702/40

(57) ABSTRACT

When the sensed image of an object has a reject, the reject reason is input. The input reject reason is stored in a storage medium in correspondence with radiographic information containing at least the sensed image.

### FIELD OF THE INVENTION

The present invention relates to a radiographic apparatus which senses an image of radiation which has passed through an object and outputs the sensed image, and a control method and program thereof.

### BACKGROUND OF THE INVENTION

When a certain kind of phosphor is irradiated with radiation (e.g., X-rays, α-rays, β-rays, γ-rays, electron beam, or UV rays), part of the radiation energy is accumulated in the phosphor. When the phosphor is irradiated with excitation light such as visible light, it exhibits photostimulated luminescence in accordance with the accumulated radiation energy, as is known. The phosphor having such a characteristic is called an accumulative phosphor (stimulable phosphor).

Using this accumulative phosphor, radiographic image information of an object such as a human body is temporarily recorded on an accumulative phosphor sheet. The accumulative phosphor sheet is scanned by excitation light such as a laser beam and caused to emit stimulable light. The obtained stimulable light is photoelectrically read to obtain an image signal.

Radiation image recording and read-pit apparatus has been proposed, which outputs the radiographic image of an object to a recording material such as a photosensitive material or a display device such as a CRT as a visible image on the basis of the image signal (e.g., Japanese Patent Laid-Open Nos. 55-12429 and 56-11395).

In recent years, systems using a digital X-ray imaging apparatus which senses an X-ray image by using a semiconductor sensor have been developed. These systems are practically advantageous because an image can be recorded over a much wider radiation exposure range as compared to conventional radiographic systems using silver-halide photography.

More specifically, X-rays in a very wide dynamic range are read by a photoelectric conversion means and converted into an electrical signal. By using this electrical signal, a radiographic image is output to a recording material such as a photosensitive material or a display device such as a CRT as a visible image. Accordingly, a radiographic image free from the influence of a variation in radiation exposure can be obtained.

In the conventional film-screen system, if the transmitted X-ray dose is insufficient, sufficient blackening cannot be obtained on the film. If the transmitted X-ray dose is too large, the film blackens excessively. That is, an error film unsuitable for observation may be generated. Such an error film is called a reject. A reject occurs not only when the transmitted X-ray dose is too large or too small but also when the patient as the object has carelessly moved.

Films with rejects are not used for diagnosis and are placed together in a storage unit such as a box. At the end of the month, the number of reject films in the storage unit is counted and used to totalize the number of used films. Although the number of reject films is counted in this work, they are not totalized for each reject reason or operator.

In the digital X-ray imaging apparatus, even when a reject has occurred, the number of rejects cannot be counted unless, e.g., they are output to films. In addition, since the images of the rejects are not transferred to the image server, the record of rejects is not made.

### SUMMARY OF THE INVENTION

The present invention has been made in consideration of the above-described problems, and has as its object to provide a radiographic apparatus which can easily confirm the reject reason of each radiographic image and provide information to efficiently suppress occurrence of rejects at the time of radiography, a control method and program thereof.

According to the present invention, the foregoing object is attained by providing a radiographic apparatus which senses an image of radiation which has passed through an object and outputs a sensed image, comprising:
reception means for receiving a reject reason when the sensed image of the object has a reject; and
storage means for storing the reject reason received by the reception means in a storage medium.

According to the present invention, the foregoing object is attained by providing a radiographic apparatus which senses an image of radiation which has passed through an object and outputs a sensed image, comprising:
input means for inputting a reject reason when the sensed image of the object has a reject; and
storage means for storing the reject reason input by the input means in a storage medium in correspondence with radiographic information containing at least the sensed image.

In a preferred embodiment, the storage means stores the reject reason and the radiographic information in the storage medium for each operator of the radiographic apparatus.

In a preferred embodiment, the radiographic information contains radiographic condition information of the sensed image.

In a preferred embodiment, the radiographic information contains object information about the object corresponding to the sensed image.

In a preferred embodiment, the apparatus further comprises display means for displaying a reject reason candidate for the sensed image.

In a preferred embodiment, the display means displays an occurrence frequency for each reject reason.

In a preferred embodiment, the display means displays the reject reason for each operator of the radiographic apparatus.

In a preferred embodiment, the display means displays the reject reason for each radiographic condition of the sensed image.

In a preferred embodiment, the display means displays the reject reason for each object information of the object corresponding to the sensed image.

According to the present invention, the foregoing object is attained by providing a radiographic apparatus which senses an image of radiation which has passed through an object and outputs a sensed image, comprising:
storage means for storing a reject reason of the sensed image of the object in correspondence with radiographic information containing at least the sensed image; and
display means for displaying the reject reason of the sensed image stored by the storage means before radiographing a new object.

In a preferred embodiment, the display means displays an occurrence frequency for each reject reason.

In a preferred embodiment, the display means displays the reject reason for each operator of the radiographic apparatus.

In a preferred embodiment, the display means displays the reject reason for each radiographic condition of the sensed image.

In a preferred embodiment, the display means displays the reject reason for each object information of the object corresponding to the sensed image.

According to the present invention, the foregoing object is attained by providing a control method of a radiographic apparatus which senses an image of radiation which has passed through an object and outputs a sensed image, comprising:
a reception step of receiving a reject reason when the sensed image of the object has a reject; and
a storage step of storing the reject reason received in the reception step in a storage medium.

According to the present invention, the foregoing object is attained by providing a control method of a radiographic apparatus which senses an image of radiation which has passed through an object and outputs a sensed image, comprising:
an input step of inputting a reject reason when the sensed image of the object has a reject; and
a storage step of storing the reject reason input in the input step in a storage medium in correspondence with radiographic information containing at least the sensed image.

According to the present invention, the foregoing object is attained by providing a control method of a radiographic apparatus which senses an image of radiation which has passed through an object and outputs a sensed image, comprising:
a storage step of storing a reject reason of the sensed image of the object in a storage medium in correspondence with radiographic information containing at least the sensed image; and
a display step of displaying the reject reason of the sensed image stored in the storage medium before radiographing a new object.

According to the present invention, the foregoing object is attained by providing a program which implements control of a radiographic apparatus which senses an image of radiation which has passed through an object and outputs a sensed image, comprising:
a program code for a reception step of receiving a reject reason when the sensed image of the object has a reject; and
a program code for a storage step of storing the reject reason received in the reception step in a storage medium.

According to the present invention, the foregoing object is attained by providing a program which implements control of a radiographic apparatus which senses an image of radiation which has passed through an object and outputs a sensed image, comprising:
a program code for an input step of inputting a reject reason when the sensed image of the object has a reject; and
a program code for a storage step of storing the reject reason input in the input step in a storage medium in correspondence with radiographic information containing at least the sensed image.

According to the present invention, the foregoing object is attained by providing a program which implements control of a radiographic apparatus which senses an image of radiation which has passed through an object and outputs a sensed image, comprising:
a program code for a storage step of storing a reject reason of the sensed image of the object in a storage medium in correspondence with radiographic information containing at least the sensed image; and
a program code for a display step of displaying the reject reason of the sensed image stored in the storage medium before radiographing a new object.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

The accompanying drawings, which are incorporated in and constitute a part of the specification, illustrate embodiments of the invention and, together with the description, serve to explain the principles of the invention.

FIG. 1 is a view showing an example of the system configuration of an X-ray imaging apparatus according to the embodiment of the present invention;

FIG. 2 is a view showing an example of an operation window according to the embodiment of the present invention;

FIG. 3 is a view showing an example of an image confirmation window according to the embodiment of the present invention;

FIG. 4 is a view showing an example of a reject reason selection window according to the embodiment of the present invention;

FIG. 5 is a view showing an example of a reject reason statistic display window according to the embodiment of the present invention; and

FIG. 6 is a flowchart showing processing executed by the X-ray imaging apparatus according to the embodiment of the present invention.

### DETAILED DESCRIPTION OF THE PREFERRED EMBODIMENT

A preferred embodiment of the present invention will be described in detail in accordance with the accompanying drawings.

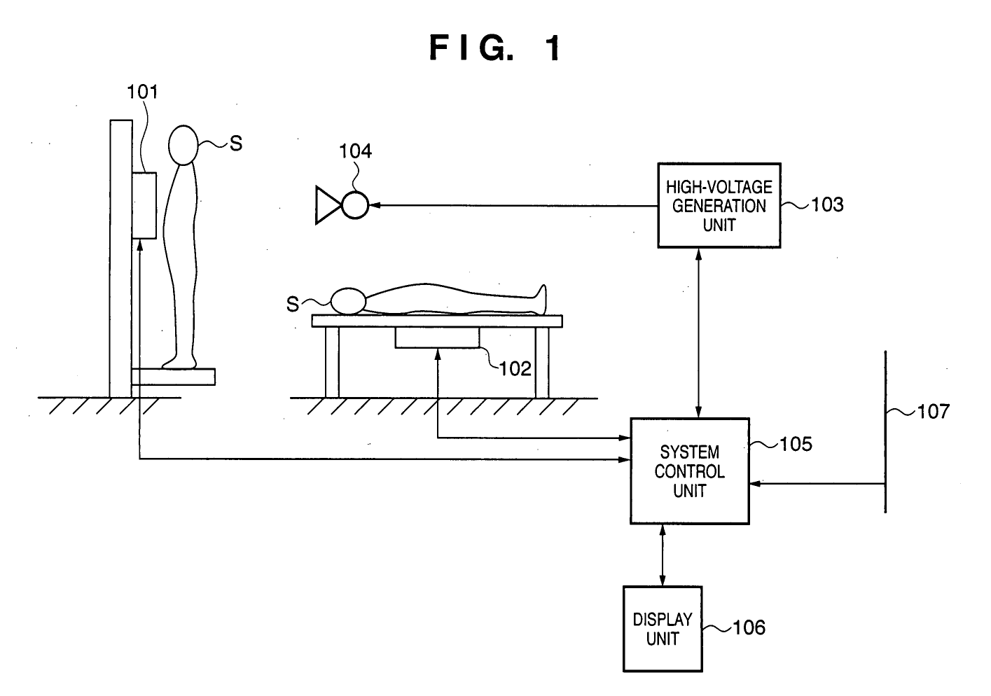

FIG. 1 is a view showing an example of the system configuration of an X-ray imaging apparatus according to the embodiment of the present invention.

Arrows in FIG. 1 indicate the flows of information and commands.

Referring to FIG. 1, the X-ray imaging apparatus comprises a standing position sensor unit 101, lying position sensor unit 102, high-voltage generation unit 103, X-ray tube 104, system control unit 105, and display unit (e.g., a CRT or LCD) 106.

Reference numeral 107 denotes a network. The X-ray imaging apparatus is connected to an HIS (Hospital Information System)/RIS (Radiology Information System) server (not shown) or an image management server (not shown) on the network 107 through the network 107.

The HIS/RIS server functions as a database server which transmits radiographic condition information or object information related to radiography by the X-ray imaging apparatus to the X-ray imaging apparatus and manages radiography execution results by the X-ray imaging apparatus.

The network 107 is a so-called communication network typically implemented by the Internet, LAN, WAN, telephone line, leased digital line, ATM, frame relay line, communication satellite channel, cable TV line, or data broadcast radio channel or a combination thereof. The network 107 only needs to be able to transmit/receive data.

The system control unit 105 controls the radiographic sequence related to the standing position sensor unit 101, lying position sensor unit 102, high-voltage generation unit 103, and X-ray imaging apparatus. The system control unit 105 has, e.g., the constituent elements of a general-purpose computer (e.g., a CPU, a memory (RAM or ROM), hard disk, external storage unit (CD-ROM drive or DVD-ROM/RAM drive), and network interface).

The standing position sensor unit 101 and lying position sensor unit 102 A/D-convert charges corresponding to the dose of X-rays which have passed through an object S and transfer the charges to the system control unit 105 as an electronic image. The object S will be referred to as a patient hereinafter as needed.

The display unit 106 displays a graphic user interface to make the operator execute an operation for setting related to the high-voltage generation unit 103 or X-ray imaging. In an operation window provided by the graphic user interface, object information containing the name, sex, and age of the patient (object S) and radiographic information containing a sensed image are displayed.

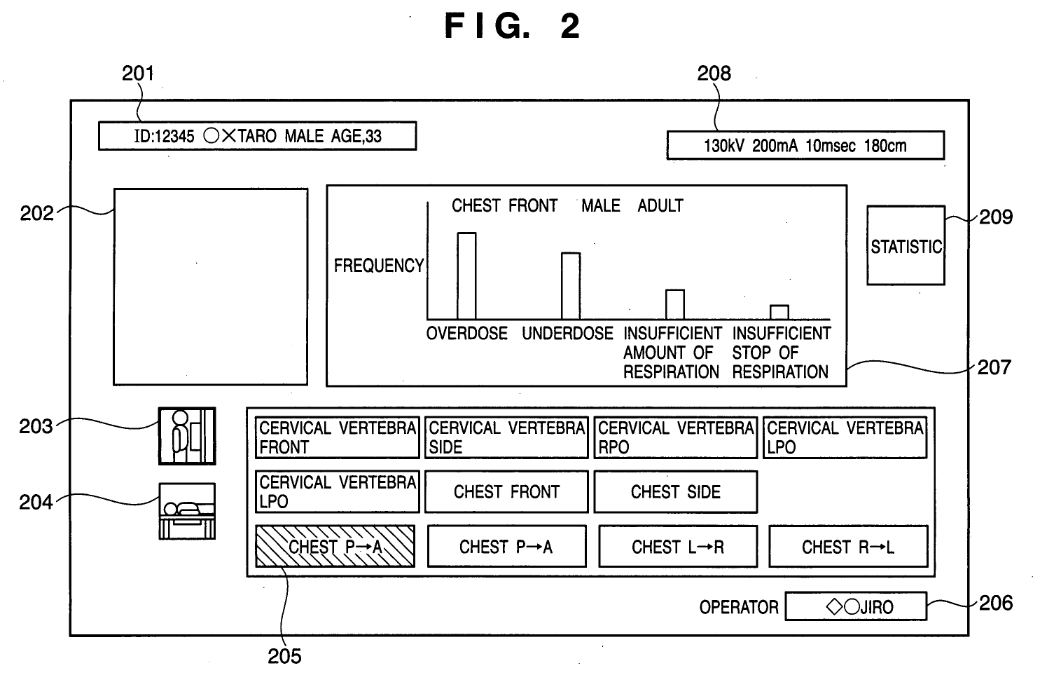

An example of the operation window displayed on the display unit 106 will be described with reference to FIG. 2.

FIG. 2 is a view showing an example of the operation window according to the embodiment of the present invention.

A touch panel including, e.g., a liquid crystal display and an analog resistive film touch sensor sheet is applied to the display unit 106 of this embodiment. The display unit 106 displays a sensed image in a sensed image region 202. In the example shown in FIG. 2, no sensed image is displayed because radiography is not done yet. If the object image is taken from the front and side in chest radiography, the sensed image of the chest front is obtained first and displayed in the sensed image region 202.

An object information display region 201 displays object information (patient information) received from an external device (e.g., HIS/RIS server). In the object information display region 201, object information containing, e.g., the name, sex, age, and ID of the patient is displayed.

A standing position sensor unit button 203 is pressed to switch the sensor to the standing position sensor unit 101. A lying position sensor unit button 204 is pressed to switch the sensor to the lying position sensor unit 102. When the standing position sensor unit button 203 is pressed, the sensor of the standing position sensor unit 101 is set in a ready state, and a radiography menu 205 (to be described later) changes to that for standing position radiography. On the other hand, when the lying position sensor unit button 204 is pressed, the sensor of the lying position sensor unit 102 is set in a ready state, and the radiography menu 205 (to be described later) changes to that for lying position radiography.

The radiography menu 205 includes body part buttons to designate various kinds of radiography target body parts. In the example shown in FIG. 2, the “chest P→A” button is selected, i.e., the chest front image should be taken. When an arbitrary one of the body parts buttons on the radiography menu is pressed, a desired body part is selected. When a body part button is pressed, display in a reject reason display region 207 changes.

An operator name display/selection region 206 displays an operator. The operator name display/selection region 206 is formed as, e.g., a pull-down menu. When an arbitrary operator is selected from the pull-down menu, the operator name can be changed. If the operator name is to be changed, a password input window to input a password is displayed to confirm whether it is an authentic operator.

The reject reason display region 207 displays, e.g., reject reasons for sensed images in the past which are classified by the radiographic condition, body part, sex, age, and operator. In this example, the reject reasons are extracted on the basis of a category including “chest front” (body part), “male” (sex), and “adult” (age). The reject reasons such as an overdose/underdose and excessive/insufficient amount of respiration of the patient are displayed before radiography to call operator's attention to prevent any further reject.

Reference numeral 208 denotes a radiographic condition display region. As the radiographic conditions, the tube voltage, tube current, exposure time, and radiographic distance are set in advance for each body part. When an arbitrary body part button is selected from the radiography menu 205, the set values of corresponding radiographic conditions are displayed in the radiographic condition display region 208.

A statistic window transition button 209 is pressed for transition to a statistic window in which statistics are displayed in detail. The statistic window can display the reject reasons for, e.g., each operator. This will be described later in detail.

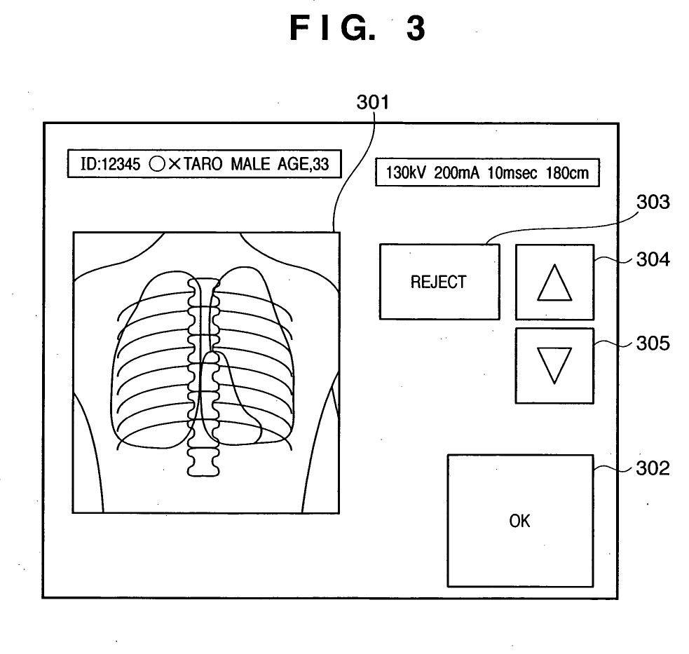

An example of an image confirmation window displayed in the sensed image region 202 of the display unit 106 after radiography will be described next with reference to FIG. 3.

FIG. 3 is a view showing an example of the image confirmation window according to the embodiment of the present invention.

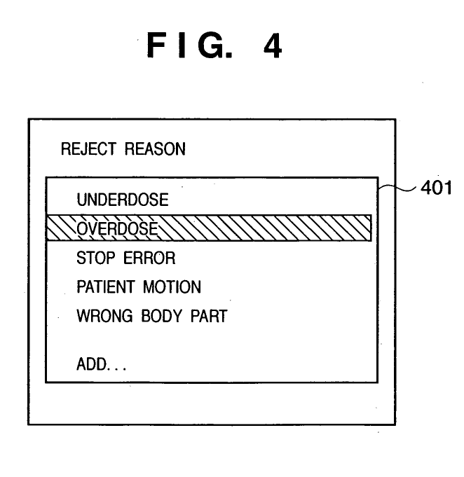

A sensed image display region 301 displays a sensed image. When the operator confirms the sensed image, and the image has no problem, the sensed image is determined by pressing an OK button 302 and stored in, e.g., the hard disk in the system control unit 105. On the other hand, if it is determined that there is a reject reason such as an underdose, overdose, or motion of the patient, the operator presses a reject button 303. In this case, a reject reason selection window (FIG. 4) is displayed to select a reject.

A preceding image selection button 304 is used to select an image taken before the sensed image displayed in the sensed image display region 301 and display the selected image in the sensed image display region 301. A succeeding image selection image 305 is used to select an image taken after the sensed image displayed in the sensed image display region 301 and display the selected image in the sensed image display region 301.

Even when the OK button 302 is temporarily pressed for sensed images at the time of radiography, the reject button 303 can appropriately be pressed again for a desired sensed image selected by using the image selection buttons. In addition, when the reject button 303 is pressed for a sensed image, and the operator is going to end radiography without selecting any reject reason in the reject reason selection window (FIG. 4), a dialogue to call operator's attention to select a reject reason is displayed.

An example of the reject reason selection window which is displayed to select a reject reason when the reject button 303 is pressed will be described next with reference to FIG. 4.

FIG. 4 is a view showing an example of a reject reason selection window according to the embodiment of the present invention.

A reject reason selection menu 401 displays a plurality of kinds of reject reason candidates. The operator can input the final reject reason by selecting an arbitrary reject reason candidate. If no reject reason candidate is present in the reject reason selection menu 401, a reject reason candidate can additionally be registered. Especially, additional registration of a reject reason can be implemented by, e.g., inputting characters from the keyboard by using a dedicated text input box.

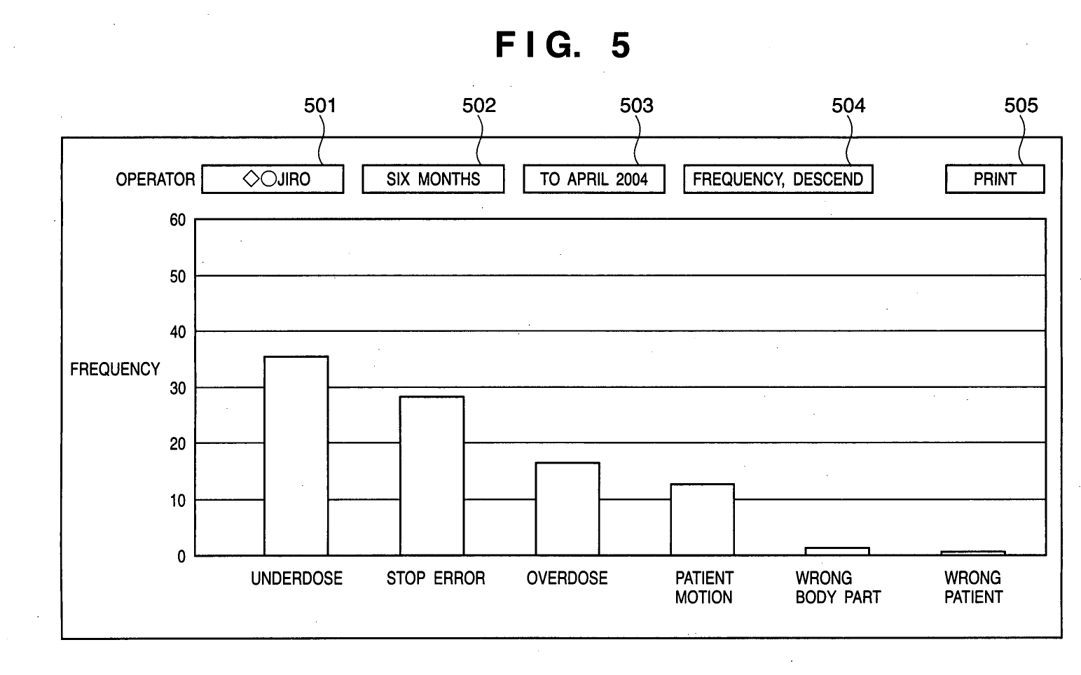

An example of a reject reason statistic display window displayed in the reject reason display region 207 will be described next with reference to FIG. 5.

FIG. 5 is a view showing an example of a reject reason statistic display window according to the embodiment of the present invention.

In the example shown in FIG. 2, the reject reasons are classified by the radiographic condition, body part, sex, and age and displayed before radiography. In the example shown in FIG. 5, the reject reasons are displayed for each operator.

An operator name display/selection region 501 is formed as a pull-down menu to select an operator. The name of an operator of the X-ray imaging apparatus can arbitrarily be selected in this window. When all operators are selected, the statistic of reject reasons for all operators is displayed.

A period selection region 502 includes a menu to set the statistic period as the range of statistic of reject reasons. A plurality of kinds of predetermined period units such as one month, three months, six months, and one year can be set as the statistic period.

A date selection region 503 includes a menu to set the date of statistic compilation.

A display method selection region 504 includes a menu to select the reject reason display method. In this example, the reject reasons are displayed in descending order of occurrence frequency. Recent reject images may be displayed sequentially as a log. Alternatively, reject reasons for all operators may be sorted in descending order of frequency and displayed.

A print button 505 is pressed to print the reject reason statistic display window on a printing paper sheet.

In this example, the reject reason statistic display window is printed on a printing paper sheet. The statistic values displayed in the reject reason statistic display window may be output to the hard disk in the X-ray imaging apparatus or HIS/RIS server as, e.g., text data in the CSV (Comma Separate Value) format for each reject reason.

An example of processing executed by the X-ray imaging apparatus according to this embodiment will be described next.

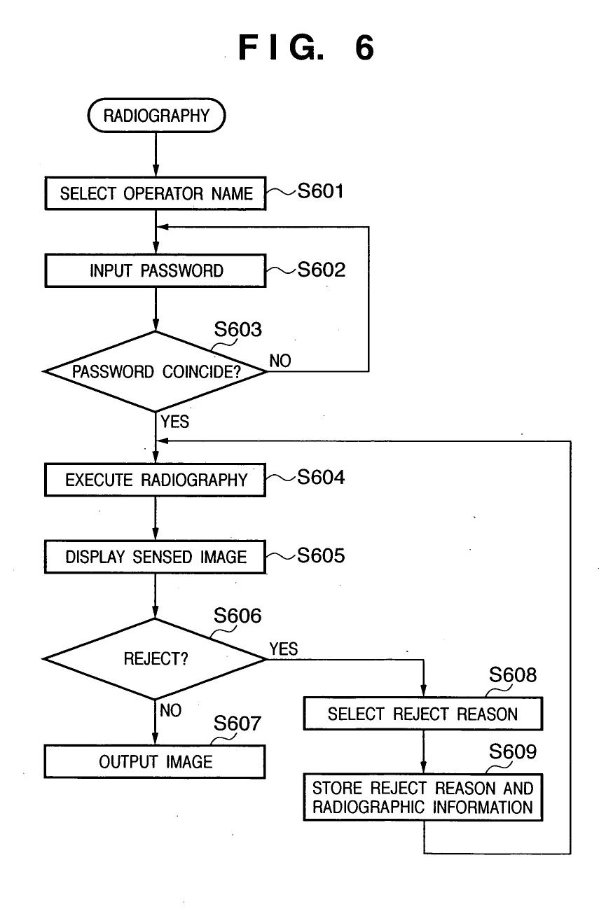

FIG. 6 is a flowchart showing processing executed by the X-ray imaging apparatus according to the embodiment of the present invention.

First, in step S601, an operator name is selected on the display unit 106 on the basis of the operator's operation in the operator name display/selection region 206 on the operation window (FIG. 2).

In step S602, after the operator name is selected, the password input window to input a password to confirm whether the operator is authentic is displayed to receive input of the password.

In step S603, it is determined whether the password registered in the memory of the system control unit 105 in advance coincides with the password input in step S602. If the passwords coincide with each other (YES in step S603), the flow advances to step S604. If the passwords do not coincide with each other (NO in step S603), the flow returns to step S602 to prompt re-input of the password.

Password re-input may be limited to a predetermined number of times (e.g., three times). If the passwords do not coincide before re-input reaches the predetermined number of times, the processing may be ended determining the operation as password input by an inauthentic operator.

In step S604, the system control unit 105 executes the radiography sequence of the patient (object S) on the basis of patient information or radiographic condition information received from the HIS/RIS server connected to the network 107.

In step S605, the standing position sensor unit 101 or lying position sensor unit 102 forms an electronic image on the basis of the dose of X-rays which have transmitted through the patient (object S). The image is processed by the system control unit 105 and displayed in the sensed image display region 301.

In step S606, the operator confirms the sensed image displayed in the sensed image display region 301 and determines whether the sensed image has a reject. The operator presses the reject button 303 or OK button 302 in accordance with the determination.

For this reason, in step S606, the presence/absence of press of the reject button 303 or OK button 302 is determined. If the OK button 302 is pressed (NO in step S606), the flow advances to step S607 to output the sensed image to the HIS/RIS server through the network 107.

If the reject button 303 is pressed (YES in step S606), the flow advances to step S608 to display the reject reason display window (FIG. 4) and receive selection of a reject reason by the operator.

In step S609, the selected reject reason and radiographic information (operator, radiographic unit (standing position sensor unit 101 or lying position sensor unit 102), sensed body part, date of radiography, radiographic condition, sex, and age) are made to correspond to each other and stored in the hard disk or memory in the system control unit 105. Since a reject has occurred, the flow returns to step S604 to execute radiography again.

In the arrangement shown in FIG. 6, when it is determined in step S606 that the reject button 303 is pressed, the reject reason selection window is displayed in step S608 to make the operator select a reject reason. Instead, the reject reason selection window (FIG. 4) may be displayed to make the operator select a reject reason after re-radiography is ended. In this arrangement, since the reject reason is selected after the end of re-radiography, the processing can be executed without interrupting the radiography. For this reason, the operation efficiency can be increased.

The reject reason statistic display window shown in FIG. 5 is generated on the basis of the reject reasons and radiographic information stored in the hard disk or memory in the system control unit 105, as a matter of course.

In step S609, the reject reason and radiographic information are made to correspond to each other and stored in the hard disk or memory in the system control unit 105. Instead, reject reasons may arbitrarily be classified by the operator, radiographic condition, or object in accordance with the application purpose or purpose and stored in the hard disk or memory in the system control unit 105.

As described above, according to this embodiment, the reject reason of a sensed image and corresponding radiographic information (e.g., the operator name) are classified and displayed. Accordingly, the operator can easily recognize the reject reason of the sensed image.

In addition, when the reject reason is presented in advance before radiography, the operator can recognize hints for the radiographic operation. Accordingly, the variation in accuracy of radiography between operators can be minimized as much as possible.

Note that the present invention can be applied to an apparatus comprising a single device or to system constituted by a plurality of devices.

Furthermore, the invention can be implemented by supplying a software program, which implements the functions of the foregoing embodiments, directly or indirectly to a system or apparatus, reading the supplied program code with a computer of the system or apparatus, and then executing the program code. In this case, so long as the system or apparatus has the functions of the program, the mode of implementation need not rely upon a program.

Accordingly, since the functions of the present invention are implemented by computer, the program code installed in the computer also implements the present invention. In other words, the claims of the present invention also cover a computer program for the purpose of implementing the functions of the present invention.

In this case, so long as the system or apparatus has the functions of the program, the program may be executed in any form, such as an object code, a program executed by an interpreter, or scrip data supplied to an operating system.

Example of storage media that can be used for supplying the program are a floppy disk, a hard disk, an optical disk, a magneto-optical disk, a CD-ROM, a CD-R, a CD-RW, a magnetic tape, a non-volatile type memory card, a ROM, and a DVD (DVD-ROM and a DVD-R).

As for the method of supplying the program, a client computer can be connected to a website on the Internet using a browser of the client computer, and the computer program of the present invention or an automatically-installable compressed file of the program can be downloaded to a recording medium such as a hard disk. Further, the program of the present invention can be supplied by dividing the program code constituting the program into a plurality of files and downloading the files from different websites. In other words, a WWW (World Wide Web) server that downloads, to multiple users, the program files that implement the functions of the present invention by computer is also covered by the claims of the present invention.

It is also possible to encrypt and store the program of the present invention on a storage medium such as a CD-ROM, distribute the storage medium to users, allow users who meet certain requirements to download decryption key information from a website via the Internet, and allow these users to decrypt the encrypted program by using the key information, whereby the program is installed in the user computer.

Besides the cases where the aforementioned functions according to the embodiments are implemented by executing the read program by computer, an operating system or the like running on the computer may perform all or a part of the actual processing so that the functions of the foregoing embodiments can be implemented by this processing.

Furthermore, after the program read from the storage medium is written to a function expansion board inserted into the computer or to a memory provided in a function expansion unit connected to the computer, a CPU or the like mounted on the function expansion board or function expansion unit performs all or a part of the actual processing so that the functions of the foregoing embodiments can be implemented by this processing.

As many apparently widely different embodiments of the present invention can be made without departing from the spirit and scope thereof, it is to be understood that the invention is not limited to the specific embodiments thereof except as defined in the appended claims.

### CLAIM OF PRIORITY

This application claims priority from Japanese Patent Application No. 2004-013106 filed on Jan. 21, 2004, the entire contents of which are hereby incorporated by reference herein.

1. A radiographic apparatus which senses an image of radiation which has passed through an object and outputs a sensed image, comprising:
reception means for receiving a reject reason when the sensed image of the object has a reject; and
storage means for storing the reject reason received by said reception means in a storage medium.

2. A radiographic apparatus which senses an image of radiation which has passed through an object and outputs a sensed image, comprising:
input means for inputting a reject reason when the sensed image of the object has a reject; and
storage means for storing the reject reason input by said input means in a storage medium in correspondence with radiographic information containing at least the sensed image.

3. The apparatus according to claim 2, wherein said storage means stores the reject reason and the radiographic information in the storage medium for each operator of the radiographic apparatus.

4. The apparatus according to claim 2, wherein the radiographic information contains radiographic condition information of the sensed image.

5. The apparatus according to claim 2, wherein the radiographic information contains object information about the object corresponding to the sensed image.

6. The apparatus according to claim 2, further comprising display means for displaying a reject reason candidate for the sensed image.

7. The apparatus according to claim 6, wherein said display means displays an occurrence frequency for each reject reason.

8. The apparatus according to claim 6, wherein said display means displays the reject reason for each operator of the radiographic apparatus.

9. The apparatus according to claim 6, wherein said display means displays the reject reason for each radiographic condition of the sensed image.

10. The apparatus according to claim 6, wherein said display means displays the reject reason for each object information of the object corresponding to the sensed image.

11. A radiographic apparatus which senses an image of radiation which has passed through an object and outputs a sensed image, comprising:
storage means for storing a reject reason of the sensed image of the object in correspondence with radiographic information containing at least the sensed image; and
display means for displaying the reject reason of the sensed image stored by said storage means before radiographing a new object.

12. The apparatus according to claim 11, wherein said display means displays an occurrence frequency for each reject reason.

13. The apparatus according to claim 11, wherein said display means displays the reject reason for each operator of the radiographic apparatus.

14. The apparatus according to claim 11, wherein said display means displays the reject reason for each radiographic condition of the sensed image.

15. The apparatus according to claim 11, wherein said display means displays the reject reason for each object information of the object corresponding to the sensed image.

16. A control method of a radiographic apparatus which senses an image of radiation which has passed through an object and outputs a sensed image, comprising:
a reception step of receiving a reject reason when the sensed image of the object has a reject; and
a storage step of storing the reject reason received in the reception step in a storage medium.

17. A control method of a radiographic apparatus which senses an image of radiation which has passed through an object and outputs a sensed image, comprising:
an input step of inputting a reject reason when the sensed image of the object has a reject; and
a storage step of storing the reject reason input in the input step in a storage medium in correspondence with radiographic information containing at least the sensed image.

18. A control method of a radiographic apparatus which senses an image of radiation which has passed through an object and outputs a sensed image, comprising:
a storage step of storing a reject reason of the sensed image of the object in a storage medium in correspondence with radiographic information containing at least the sensed image; and
a display step of displaying the reject reason of the sensed image stored in the storage medium before radiographing a new object.

19. A program which implements control of a radiographic apparatus which senses an image of radiation which has passed through an object and outputs a sensed image, comprising:
a program code for a reception step of receiving a reject reason when the sensed image of the object has a reject; and
a program code for a storage step of storing the reject reason received in the reception step in a storage medium.

20. A program which implements control of a radiographic apparatus which senses an image of radiation which has passed through an object and outputs a sensed image, comprising:
a program code for an input step of inputting a reject reason when the sensed image of the object has a reject; and
a program code for a storage step of storing the reject reason input in the input step in a storage medium in correspondence with radiographic information containing at least the sensed image.

21. A program which implements control of a radiographic apparatus which senses an image of radiation which has passed through an object and outputs a sensed image, comprising:
a program code for a storage step of storing a reject reason of the sensed image of the object in a storage medium in correspondence with radiographic information containing at least the sensed image; and
a program code for a display step of displaying the reject reason of the sensed image stored in the storage medium before radiographing a new object.
